(19) United States
(12) Patent Application Publication
(10) Pub. No.: US 2021/0045929 A1
(43) Pub. Date: Feb. 18, 2021

(54) Compression Garment Assembly

(71) Applicant: Leslie Derksen, Salem, OR (US)

(72) Inventor: Leslie Derksen, Salem, OR (US)

(21) Appl. No.: 16/540,918

(22) Filed: Aug. 14, 2019

(51) Int. Cl. A61F 13/08; A41D 1/06

(52) U.S. Cl. CPC A61F 13/08; A61H 2205/108; A41D 2400/38; A41D 1/06

(57) ABSTRACT

A compression garment assembly includes a compression garment that is worn on a user's lower body. The compression garment is comprised of an elastic material thereby facilitating the compression garment to squeeze against the user's body wherein the compression garment to inhibit the formation of varicose veins in the user's lower body. A plurality of first mating members is each integrated into the compression garment. Each of the first mating members is positioned to extend along strategic locations on the user's body when the compression garment is worn. A plurality of second mating members is each wrappable around the compression garment at strategic locations on the compression garment.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to compression devices and more particularly pertains to a new compression device for inhibiting the formation of varicose veins.

### (2) Description of Related Art Including Information Disclosed Under 37 CFR 1.97 and 1.98

The prior art relates to compression devices.

### (h) BRIEF SUMMARY OF THE INVENTION

An embodiment of the disclosure meets the needs presented above by generally comprising a compression garment that is worn on a user's lower body. The compression garment is comprised of an elastic material thereby facilitating the compression garment to squeeze against the user's body wherein the compression garment to inhibit the formation of varicose veins in the user's lower body. A plurality of first mating members is each integrated into the compression garment. Each of the first mating members is positioned to extend along strategic locations on the user's body when the compression garment is worn. A plurality of second mating members is each wrappable around the compression garment at strategic locations on the compression garment.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a compression garment assembly according to an embodiment of the disclosure.

FIG. 2 is a back perspective view of an embodiment of the disclosure.

FIG. 3 is a left side view of an embodiment of the disclosure.

FIG. 4 is a perspective in-use view of an embodiment of the disclosure.

FIG. 5 is a perspective view of an embodiment of the disclosure in a long-legged configuration.

### (j) DETAILED DESCRIPTION OF THE INVENTION

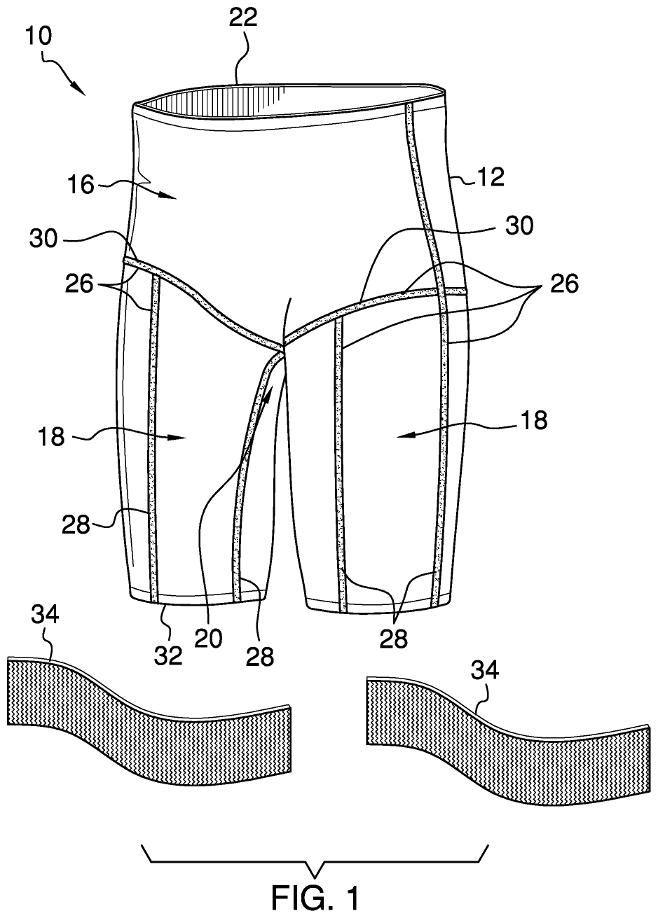

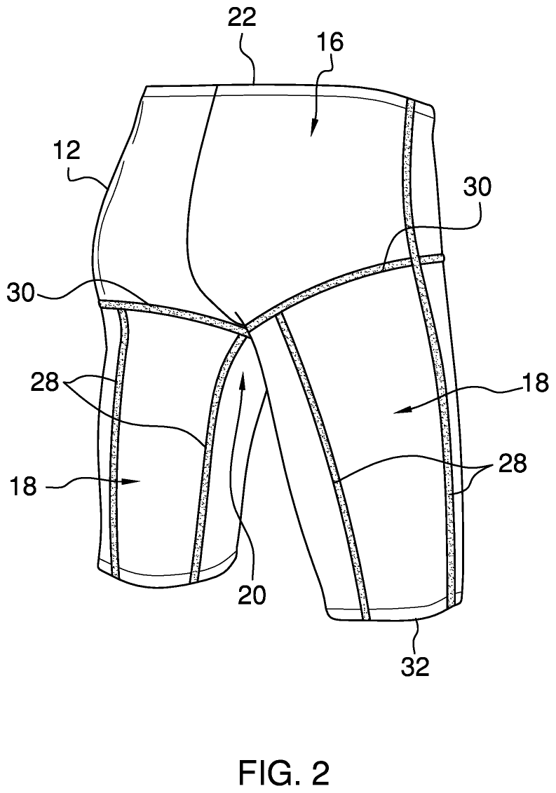

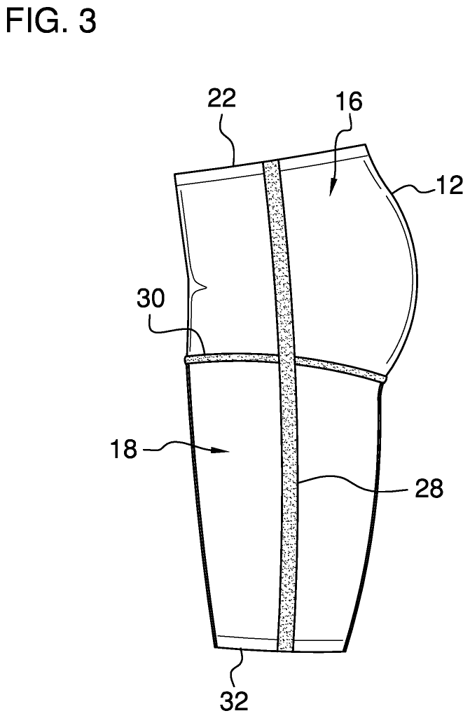

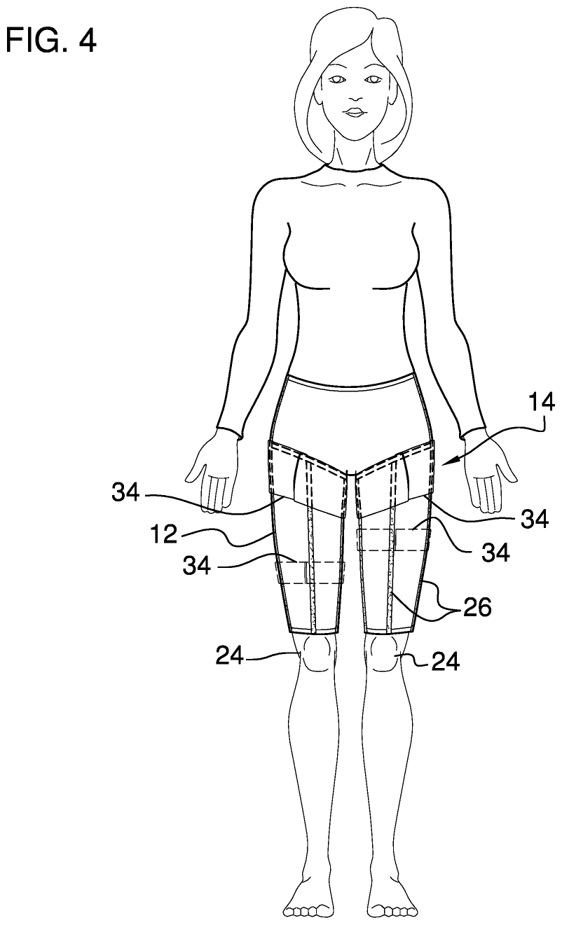

With reference now to the drawings, and in particular to FIGS. 1 through 5 thereof, a new compression device embodying the principles and concepts of an embodiment of the disclosure and generally designated by the reference numeral 10 will be described.

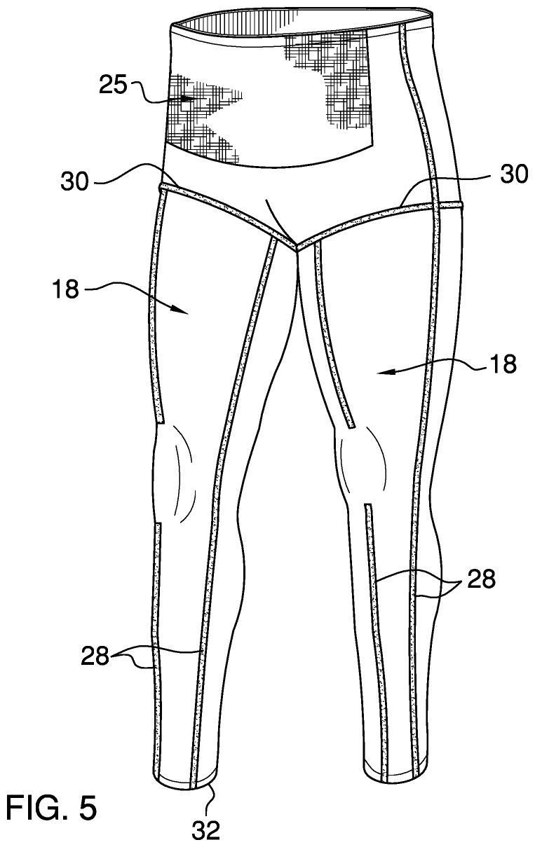

As best illustrated in FIGS. 1 through 5, the compression garment assembly 10 generally comprises a compression garment 12 that is worn on a user's lower body 14. The compression garment 12 is comprised of an elastic material thereby facilitating the compression garment 12 to squeeze against the user's lower body 14. In this way the compression garment 12 can inhibit the formation of varicose veins in the user's lower body 14. The compression garment 12 has a body portion 16, a pair of legs 18, a crotch 20 and a waist 22. As is most clearly shown in FIGS. 1 through 4, each of the legs 18 may terminate above the user's knees 24 when the compression garment 12 is worn. As is most clearly shown in FIG. 5, each of said legs 18 may terminate below the user's knees 24 when the compression garment 12 is worn. As is further shown in FIG. 5, the body portion 16 of the garment may have a stretchable panel 25 for accommodating the belly of a pregnant woman.

A plurality of first mating members 26 is each integrated into the compression garment 12. Each of the first mating members 26 is positioned to extend along strategic locations on the user's body 14 when the compression garment 12 is worn. Moreover, each of the first mating members 26 may comprise a loop portion of a set of hook and loop fasteners or the like.

The plurality of first mating members 26 includes a plurality of vertically oriented mating members 28 and a plurality of horizontally oriented mating members 30. Each of the horizontally oriented mating members 30 extends around a respective one of the legs 18. Each of the horizontally oriented mating members 30 is aligned with the crotch 20. In this way each of the horizontally oriented mating members 30 can wrap around an upper threshold of the user's thighs when the compression garment 12 is worn.

Each of the vertically oriented mating members 28 is positioned on a respective one of the legs 18. Additionally, the vertically oriented mating members 28 on each of the legs 18 are spaced from each other and are distributed around an entire circumference of the legs 18. A respective plurality of the vertically oriented mating members 28 extends between the horizontally oriented mating member 30 on the respective leg 18 and a terminal end 32 of the respective leg. A respective pair of the vertically oriented mating members 28 extends between the waist 22 and the terminal end 32 of the respective leg 18 along an outer side of the respective leg 18.

A plurality of second mating members 34 is provided and each of the second mating members 34 is wrappable around the compression garment 12 at strategic locations on the compression garment 12. Additionally, each of the second mating members 34 engages respective first mating members 26. In this way the second mating members 34 tighten the compression garment 12 around the strategic locations in the effort of inhibiting the formation of varicose veins. Each of the second mating members 34 is wrappable and tightenable around a respective one of the legs 18. Moreover, each of the second mating members 34 may comprise a hook portion of a set of hook and loop fasteners or the like. Each of the second mating members 34 is structured to form an elongated strap.

In use, the compression garment 12 is worn to inhibit the formation of varicose veins. The compression garment 12 may be worn by a woman during pregnancy, an elderly person with poor circulation or by any individual seeking relieve from varicose veins. The second mating members 34 can be wrapped around legs 18 to increase the pressure on the user's body 14. Additionally, the second mating members 34 can be positioned at strategic locations on the user's legs 18. In this way the user can apply pressure to selected areas on the legs 18 that are most vulnerable to developing varicose veins.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A compression garment assembly being configured to apply a pre-determined amount of pressure around strategic locations on a user's body, said assembly comprising:
a compression garment being worn on a user's lower body, said compression garment being comprised of an elastic material thereby facilitating said compression garment to squeeze against the user's body wherein said compression garment is configured to inhibit the formation of varicose veins in the user's lower body;
a plurality of first mating members, each of said first mating members being integrated into said compression garment, each of said first mating members being positioned to extend along strategic locations on the user's body when said compression garment is worn; and
a plurality of second mating members, each of said second mating members being wrappable around said compression garment at strategic locations on said compression garment, each of said second mating members engaging respective first mating members for tightening said compression garment around the strategic locations in the effort of inhibiting the formation of varicose veins.

2. The assembly according to claim 1, wherein:
said compression garment has a body portion, a pair of legs, a crotch and a waist; and
said plurality of first mating members includes a plurality of vertically oriented mating members and a plurality of horizontally oriented mating members.

3. The assembly according to claim 2, wherein each of said horizontally oriented mating members extends around a respective one of said legs, each of said horizontally oriented members being aligned with said crotch wherein each of said horizontally oriented members is configured to wrap around an upper threshold of the user's thighs when said compression garment is worn.

4. The assembly according to claim 3, wherein each of said vertically oriented mating members is positioned on a respective one of said legs, said vertically oriented mating members on each of said legs being spaced from each other and being distributed around an entire circumference of said legs.

5. The assembly according to claim 4, wherein a respective plurality of said vertically oriented mating members extends between said horizontally oriented mating member on said respective leg and a terminal end of said respective leg.

6. The assembly according to claim 4, wherein a respective pair of said vertically oriented mating members extends between said waist and said terminal end of said respective leg along an outer side of said respective leg.

7. The assembly according to claim 2, wherein each of said second mating members is wrappable around a respective one of said legs, each of said second mating members being tightenable around the respective leg.

8. A compression garment assembly being configured to apply a pre-determined amount of pressure around strategic locations on a user's body, said assembly comprising:
a compression garment being worn on a user's lower body, said compression garment being comprised of an elastic material thereby facilitating said compression garment to squeeze against the user's body wherein said compression garment is configured to inhibit the formation of varicose veins in the user's lower body, said compression garment having a body portion, a pair of legs, a crotch and a waist;
a plurality of first mating members, each of said first mating members being integrated into said compression garment, each of said first mating members being positioned to extend along strategic locations on the user's body when said compression garment is worn, said plurality of first mating members including a plurality of vertically oriented mating members and a plurality of horizontally oriented mating members, each of said horizontally oriented mating members extending around a respective one of said legs, each of said horizontally oriented members being aligned with said crotch wherein each of said horizontally oriented members is configured to wrap around an upper threshold of the user's thighs when said compression garment is worn, each of said vertically oriented mating members being positioned on a respective one of said legs, said vertically oriented mating members on each of said legs being spaced from each other and being distributed around an entire circumference of said legs, a respective plurality of said vertically oriented mating members extending between said horizontally oriented mating member on said respective leg and a terminal end of said respective leg, a respective pair of said vertically oriented mating members extending between said waist and said terminal end of said respective leg along an outer side of said respective leg; and
a plurality of second mating members, each of said second mating members being wrappable around said compression garment at strategic locations on said compression garment, each of said second mating members engaging respective first mating members for tightening said compression garment around the strategic locations in the effort of inhibiting the formation of varicose veins, each of said second mating members being wrappable around a respective one of said legs, each of said second mating members being tightenable around the respective leg.
